(19) United States
(12) Patent Application Publication
(10) Pub. No.: US 2021/0128170 A1
(43) Pub. Date: May 6, 2021

(54) HEMOSTASIS PROMOTING METHOD AND HEMOSTASIS ASSISTING TOOL

(71) Applicant: TERUMO KABUSHIKI KAISHA, Tokyo (JP)

(72) Inventors: Satoshi WADA, Fujinomiya-shi (JP); Sakyo FUKASAWA, Fujinomiya-shi (JP)

(73) Assignee: TERUMO KABUSHIKI KAISHA, Tokyo (JP)

(21) Appl. No.: 17/118,662

(22) Filed: Dec. 11, 2020

Related U.S. Application Data
(63) Continuation of application No. PCT/JP2019/024270, filed on Jun. 19, 2019.

(30) Foreign Application Priority Data
Jun. 20, 2018 (JP) 2018-117195
Jun. 20, 2018 (JP) 2018-117198

(51) Int. Cl. A61B 17/132; A61F 7/02

(52) U.S. Cl. CPC A61B 17/1322; A61B 2017/12004; A61F 7/02

(57) ABSTRACT

A hemostasis promoting method and a hemostasis assisting tool are capable of promoting hemostasis on a puncture site. The hemostasis promoting method is a method of promoting hemostasis on a puncture site formed in a radial artery among the radial artery and an ulnar artery that are branched from a brachial artery in an arm and that extend parallel to each other. The hemostasis promoting method makes a temperature of a body surface around the radial artery higher than a temperature of a body surface around the ulnar artery such that the temperature of the radial artery will be higher than the temperature of the ulnar artery.

### CROSS-REFERENCES TO RELATED APPLICATIONS

This application is a continuation of International Application No. PCT/JP2019/024270 filed on Jun. 19, 2019, which claims priority to Japanese Patent Application No. 2018-117195 filed on Jun. 20, 2018 and Japanese Patent Application No. 2018-117198 filed on Jun. 20, 2018 the entire content of each of which is incorporated herein by reference.

### FIELD OF THE INVENTION

The present invention generally relates to a hemostasis promoting method and a hemostasis assisting tool for promoting hemostasis on a puncture site.

### BACKGROUND DISCUSSION

In recent years, there has been performed a medical procedure in which a blood vessel in a limb is punctured by a puncture needle, an introducer sheath is introduced through the puncture site into the blood vessel, and a medical device such as a catheter is delivered through the lumen of the introducer sheath to a lesion for percutaneous diagnosis and treatment of the lesion.

After the puncture, it is a general practice to apply a compression force to the puncture site for performing hemostasis on the puncture site. For performing hemostasis on the puncture site, there is used a hemostatic device for compressing the puncture site and its peripheral area as disclosed in Japanese Patent Laid-Open No. Hei 11-244293 for example.

### SUMMARY

When a puncture site is to be stanched, it is necessary for the patient to keep the periphery of the puncture site as still as possible. Since the patient's movement is thus limited during hemostasis, there are demands that the time required for the hemostasis should be as short as possible by promoting the hemostasis on the puncture site.

Disclosed here is a hemostasis promoting method and a hemostasis assisting tool that are capable of promoting hemostasis on a puncture site.

A hemostasis promoting method for promoting hemostasis at a puncture site formed in a first blood vessel, the first blood vessel and a second vessel branching from a blood vessel in a limb and extending parallel to each other, the hemostasis promoting method comprising making a temperature of a body surface around the first blood vessel and/or a peripheral blood vessel of the first blood vessel higher than a temperature of a body surface around the second blood vessel and/or a peripheral blood vessel of the second blood vessel such that the temperature of the first blood vessel and/or the peripheral blood vessel of the first blood vessel will be higher than the temperature of the second blood vessel and/or the peripheral blood vessel of the second blood vessel.

According to another aspect, a hemostasis assisting tool is mountable on a limb of a living body for assisting hemostasis at a puncture site in a first blood vessel that extends parallel to a second blood vessel, with both the first and second blood vessels branching from a blood vessel in the limb. The hemostasis assisting tool comprises a temperature adjusting portion for making a temperature of a body surface around the first blood vessel and/or a peripheral blood vessel of the first blood vessel higher than a temperature of a body surface around the second blood vessel and/or a peripheral blood vessel of the second blood vessel such that the temperature of the first blood vessel and/or the peripheral blood vessel of the first blood vessel will be higher than the temperature of the second blood vessel and/or the peripheral blood vessel of the second blood vessel.

Another aspect involves a hemostasis promoting method for promoting hemostasis at a puncture site formed in a first blood vessel of a limb of a living body, with the first blood vessel and a second vessel branching from a blood vessel in the limb and extending parallel to one another. The hemostasis promoting method comprises: positioning a limb mount on the limb of the patient adjacent the puncture site so that the limb mount encircles the limb of the living body; and after the positioning of the limb mount on the limb of the living body adjacent the puncture site: i) applying heat to the body surface of the limb around the first blood vessel and/or a peripheral blood vessel of the first blood vessel to increase a temperature of the body surface around the first blood vessel and/or a peripheral blood vessel of the first blood vessel; and/or ii) applying cooling to the body surface of the limb around the second blood vessel and/or a peripheral blood vessel of the second blood vessel to decrease the temperature of the body surface around the second blood vessel and/or a peripheral blood vessel of the second blood vessel so that the temperature of the first blood vessel and/or the peripheral blood vessel of the first blood vessel is greater than the temperature of the second blood vessel and/or the peripheral blood vessel of the second blood vessel to thus cause a volume of blood flow in the first blood vessel to be increased and an amount of a blood coagulation factor in the first blood vessel to be increased so that hemostasis on the puncture site is promoted. The applying of the heat and/or the applying of the cooling is accomplished by a part on the limb mount.

The hemostasis promoting method and the hemostasis assisting tool are capable of promoting hemostasis on a puncture site.

### BRIEF DESCRIPTION OF THE DRAWINGS

FIG. 1 is a view illustrating a manner in which a hemostasis assisting tool according to a first embodiment is mounted on a forearm.

FIG. 2 is a schematic view illustrating a region where the hemostasis assisting tool according to the first embodiment develops a temperature difference.

FIG. 3 is a cross-sectional view taken along the section line 3-3 of FIG. 1.

FIG. 4 is a flowchart of a hemostasis promoting method according to an embodiment.

FIG. 5A is a schematic view illustrating a modification of a region where a temperature difference is developed.

FIG. 5B is a schematic view illustrating a modification of a region where a temperature difference is developed.

FIG. 6A is a schematic view illustrating a modification of a heating process and a cooling process.

FIG. 6B is a schematic view illustrating a modification of a heating process and a cooling process.

FIG. 7 is a plan view of a hemostasis assisting tool according to a second embodiment.

FIG. 8 is a schematic view illustrating a heated region and a cooled region developed by the hemostasis assisting tool according to the second embodiment.

FIG. 9 is a cross-sectional view taken along the section line 9-9 of FIG. 7.

FIG. 10 is a plan view of a hemostasis assisting tool according to a third embodiment.

FIG. 11A is a perspective view illustrating a manner in which the hemostasis assisting tool according to the third embodiment is mounted on a forearm.

FIG. 11B is a schematic view illustrating a heated region and a cooled region developed by the hemostasis assisting tool according to the third embodiment.

FIG. 12 is a cross-sectional view taken along the section line 12-12 of FIG. 11A.

FIG. 13 is a plan view of a hemostasis assisting tool according to a fourth embodiment.

FIG. 14 is a cross-sectional view taken along the section line 14-14 of FIG. 13.

### DETAILED DESCRIPTION

Set forth below with reference to the accompanying drawings is a detailed description of embodiments, and modifications thereof, of a hemostasis promoting method and a hemostasis assisting tool representing examples of the inventive hemostasis promoting method and hemostasis assisting tool disclosed here. The dimensions or scales on the drawings may be exaggerated or different from actuality/reality for convenience of description and illustration. The description set forth below does not limit the technical scope and the significances of the terms described in the scope of the claims.

### First Embodiment

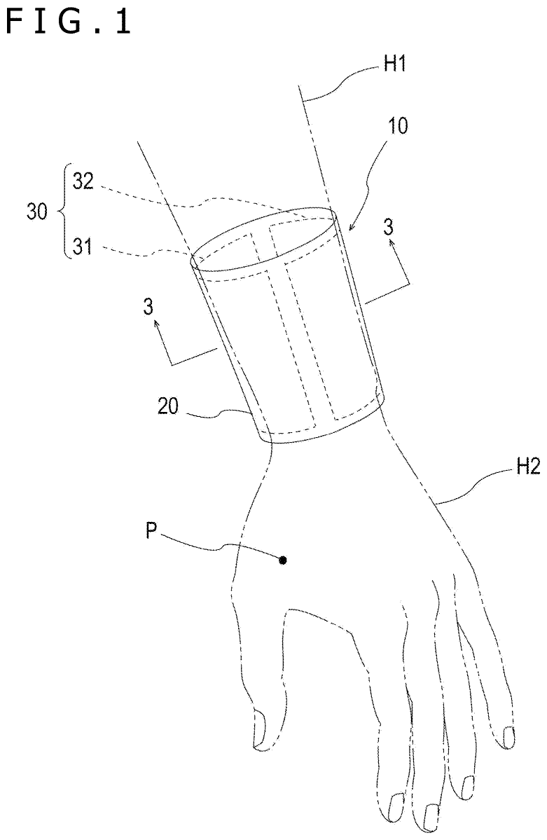

FIGS. 1 through 3 are views illustrating a hemostasis assisting tool 10 according to a first embodiment disclosed here as one example of the inventive hemostasis assisting tool 10.

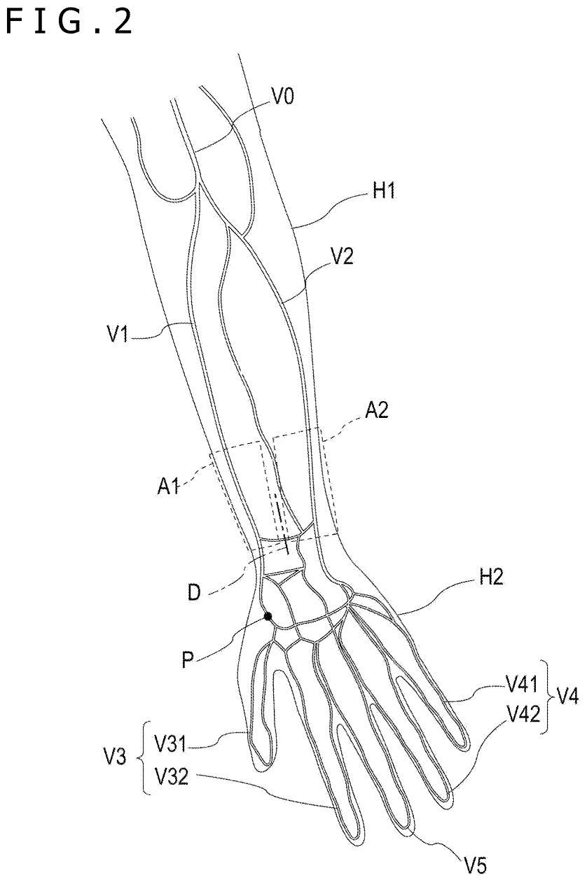

As illustrated in FIG. 2, the hemostasis assisting tool 10 according to the present embodiment assists in stanching a puncture site P formed in a radial artery V1 among the radial artery V1 (corresponding to a first blood vessel) and an ulnar artery V2 (corresponding to a second blood vessel) that are branched from a brachial artery V0 (corresponding to a blood vessel) in an arm (corresponding to a limb) and that extend parallel to each other.

The puncture site P may be formed in a radial artery in the snuffbox of a hand H2, may be formed in a distal radial artery that is closer to a fingertip than the snuffbox, or may be formed in a radial artery around a wrist. The radial artery in the snuffbox here refers to a radial artery in a region positioned between an extensor pollicis brevis muscle and an extensor pollicis longus muscle on a peripheral side of radial artery (a radial artery in the anatomical snuffbox, Netter's Atlas of Human Anatomy, 4th edition) and will hereinafter be referred to as “s-RA.” The distal radial artery refers to a radial artery positioned between an extensor pollicis brevis muscle and an extensor carpi radialis longus muscle as a dorsal carpal branch of radial artery, and will hereinafter be referred to as “d-RA.”

According to the first embodiment, the hand H2 will be described as a left hand, with the puncture site P formed in the d-RA of the patient's left hand. However, the puncture site P may be formed in the patient's right hand.

The hemostasis assisting tool 10 according to the present embodiment will generally be described below with reference to FIGS. 1 and 2. The hemostasis assisting tool 10 has a mount portion (limb mount) 20 to be mounted on an arm and a temperature adjusting portion (temperature adjuster) 30 disposed on the mount portion 20, for making a temperature of a body surface around the radial artery V1 higher than a temperature of a body surface around the ulnar artery V2 such that the temperature of the radial artery V1 will be higher than the temperature of the ulnar artery V2 while the mount portion 20 is mounted on the arm. The phrase “the temperature of the radial artery V1 will be higher than the temperature of the ulnar artery V2” here means that the temperature of the radial artery V1 will be made higher than the temperature of the ulnar artery V2 to the extent that the volume of a blood flow in the radial artery V1 will be increased. In other words, increasing the temperature of the radial artery V1 increases the volume of blood flow in the in the radial artery V1. Parts of the hemostasis assisting tool 10 will hereinafter be described in detail.

First, the mount portion 20 will be described below.

According to the present embodiment, as illustrated in FIG. 1, the mount portion 20 is constructed as a tubular member (cylindrical sleeve) through which a forearm H1 can be inserted. When the forearm H1 is inserted through the mount portion 20, the hemostasis assisting tool 10 is mounted on the forearm H1. The mount portion 20 is not limited to the above configuration. The mount portion 20 may be constructed as a band body (band) that can be wound around the forearm H1, instead of as the tubular member, for example.

The mount portion 20 should be able to heat or cool the surface of the arm while being secured to the surface of the arm and may have an outer side made of a material that can be wound around the arm. That is, the mount portion 20 should be configured in a manner allowing the surface of the arm to be heated or cooled while the mount portion 20 is secured to the surface of the arm. For example, the material may be any of various thermoplastic elastomers or the like including a styrene-based elastomer, a polyolefin-based elastomer, a polyurethane-based elastomer, a polyester-based elastomer, a polyimide-based elastomer, a polybutadiene-based elastomer, a trans-polyisoprene-based elastomer, a fluororubber-based elastomer, chlorinated polyethylene-based elastomer, etc., and a combination of one or two or more of these elastomers as a polymer alloy, a polymer blend, a layered body, or the like may be used.

The mount portion 20 may be made of a combination of a relatively hard material and a flexible material. The flexible material may be any of various rubber materials including natural rubber, isoprene rubber, butadiene rubber, chloroprene rubber, silicone rubber, fluororubber, styrene-butadiene rubber, etc., or a resin material such as any of various thermoplastic elastomers including a styrene-based elastomer, a polyolefin-based elastomer, a polyurethane-based elastomer, a polyester-based elastomer, a polyimide-based elastomer, a polybutadiene-based elastomer, a trans-polyisoprene-based elastomer, a fluororubber-based elastomer, chlorinated polyethylene-based elastomer, etc.

The mount portion 20 may be made of an existing textile material and may be made of cotton, hemp, wool fabric, nylon, polyester, acryl, or a mixture of these fibers.

The mount portion 20 may be made of a metal material for giving itself strength and flexibility. The metal material may be included as piano wire, stainless steel wire, cobalt-chromium alloy, nickel-titanium alloy, and, in addition, copper wire that is of high thermal conductivity, etc., for promoting heat exchange between the heat generating portion or the heat absorbing portion and the surface of the hand. The metal material may be a filamentary material of a reinforcing wire for increased strength or may be shaped as a plate for heat exchange.

The mount portion 20 may be shaped as a perforated or slit plate, thereby providing both flexibility and heat exchange capability.

Next, the temperature adjusting portion 30 will be described below.

According to the present embodiment, as illustrated in FIGS. 1 and 2, the temperature adjusting portion 30 has a heating portion (heater) 31 for heating a body surface around the radial artery V1 and a cooling portion (cooler) 32 for cooling a body surface around the ulnar artery V2. Parts of the temperature adjusting portion 30 will hereinafter be described in detail.

The heating portion 31 is not limited to any particular detail as long as it is capable of heating the body surface around the radial artery V1 such that the temperature of the radial artery V1 will be higher than the temperature of the ulnar artery V2. The heating portion 31 may include, for example, an electric heating device such as a heating portion of a Peltier device or an electric heating wire, a circulating-type heating device using a fluid such as warm water, a heat storage medium such as a heating pad, or the like.

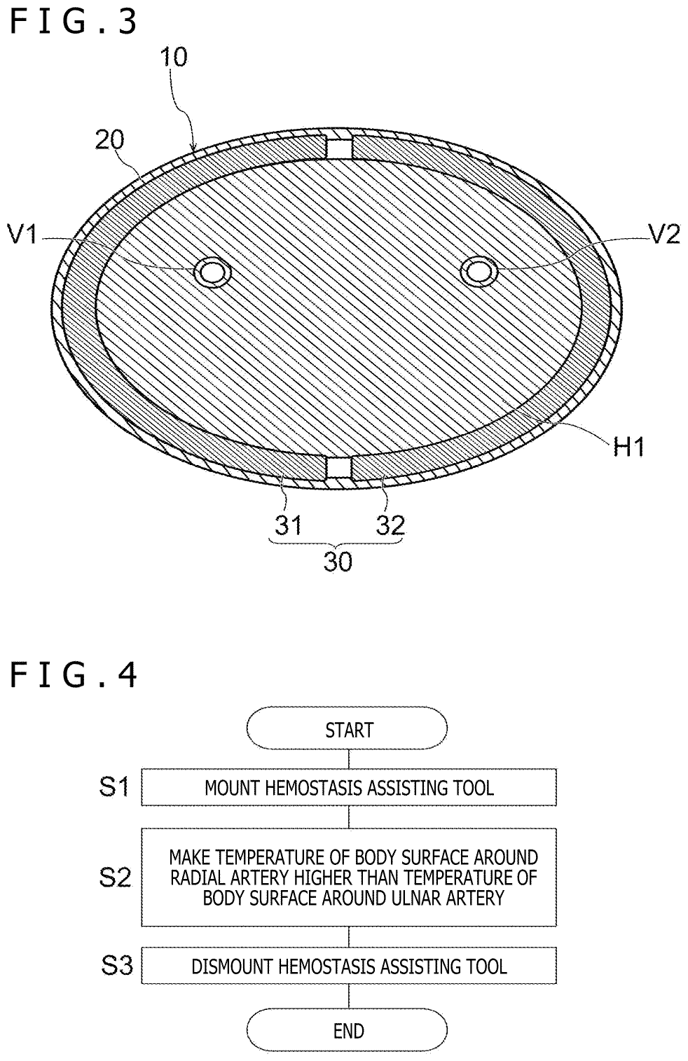

According to the present embodiment, as illustrated in FIG. 3, the heating portion 31 is disposed on an inner surface of the mount portion 20. However, the heating portion 31 may be disposed on an outer surface (a surface that faces outwardly in a mounted state) of the mount portion 20.

According to the present embodiment, as illustrated in FIG. 2, a heated region A1 of the body surface that is heated by the heating portion 31 is a region A1 of the forearm H1 that is more to the thumb side than a palmaris longus muscle D (a substantially half region of the forearm H1 on the thumb side). However, the heated region A1 is not limited to any particular region as long as it includes the body surface around the radial artery V1. The phrase “the body surface around the radial artery V1” here means a region of the body surface where heat from the heating portion 31 can be transmitted to the radial artery V1 to increase the temperature of the radial artery V1. Therefore, the heated region A1 may be, for example, only a region of the body surface of the forearm H1 that is positioned directly above the radial artery V1 when the forearm H1 is viewed in plan from the palmar side (or from the back of the hand H2).

The heating portion 31 should preferably, but not necessarily, be able to increase the temperature of the body surface up to a temperature higher than the average body surface temperature around the puncture site P of the patient (for example, the average of body temperatures measured for several minutes at the hand H2, the arm, the armpit, etc., prior to the adjustment of the temperature). The heating portion 31 should preferably, but not necessarily, be able to increase the temperature of the body surface up to a range of 33° C. to 37° C.

The cooling portion 32 is not limited to any particular detail as long as it is capable of cooling the body surface around the ulnar artery V2 such that the temperature of the ulnar artery V2 will be lower than the temperature of the radial artery V1. The cooling portion 32 may include, for example, an electric cooling device such as a heat absorbing section of a Peltier device, a circulating-type cooling device using a fluid such as cold water, a cold storage medium such as a cold pack, or the like.

According to the present embodiment, as illustrated in FIG. 3, the cooling portion 32 is disposed on an inner surface of the mount portion 20. However, the cooling portion 32 may be disposed on an outer surface (a surface that faces outwardly in a mounted state) of the mount portion 20.

According to the present embodiment, as illustrated in FIG. 2, a cooled region A2 of the body surface that is cooled by the cooling portion 32 is a region A2 of the forearm H1 that is on the little finger side of the palmaris longus muscle D as a boundary (a substantially half region of the forearm H1 on the little finger side). However, the cooled region A2 is not limited to any particular region as long as it includes the body surface around the ulnar artery V2. Note that the phrase “the body surface around the ulnar artery V2” here means a region of the body surface where heat from the ulnar artery V2 can be transmitted to the cooling portion 32 to reduce the temperature of the ulnar artery V2. Therefore, the cooled region A2 may be, for example, only a region of the body surface of the forearm H1 that is positioned directly above the ulnar artery V2 when the forearm H1 is viewed in plan from the palmar side (or from the back of the hand H2).

The cooling portion 32 should preferably, but not necessarily, be able to reduce the temperature of the body surface down to a temperature lower than the average body surface temperature around the puncture site P of the patient (for example, the average of body temperatures measured for several minutes at the hand H2, the arm, the armpit, etc.). The cooling portion 32 should preferably, but not necessarily, be able to reduce the temperature of the body surface down to a range of 10° C. to 27° C.

As well as the resin material referred to above that may be used to fabricate the mount portion 20, a metal material may be disposed between the heating portion 31 or the cooling portion 32 and the body surface for giving itself strength and flexibility. The metal material may be included as piano wire, stainless steel wire, cobalt-chromium alloy, nickel-titanium alloy, and, in addition, copper wire that is of high thermal conductivity, etc., for promoting heat exchange between the heat generating portion or the heat absorbing portion and the surface of the hand.

The metal material may be a filamentary material of a reinforcing wire for increased strength or may be shaped as a plate for heat exchange. Alternatively, the metal material may be shaped as a wound, coiled, or woven filamentary wire or may be shaped as a perforated or slit plate, thereby providing both flexibility and heat exchange capability.

The metal material may be held in direct contact with the heating portion 31 or the cooling portion 32 or may have the above resin material disposed between itself and the heating portion 31 or the cooling portion 32. Alternatively, the metal material may be held in direct contact with the body surface, or the above resin material may be disposed between the body surface and the metal material.

In order to prevent overheating or overcooling, it is preferable to provide the above resin material to a thickness ranging from approximately 0.1 to 1 mm between the body surface and the metal material.

The resin material may be of a sheet, woven, or sponge form, but should desirably incorporate fiber woven therein for transpiration in order to suppress discomfort from sweating due to heating and condensation due to cooling.

(Hemostasis Promoting Method)

FIG. 4 is a diagram illustrating a hemostasis promoting method according to an embodiment disclosed by way of example.

The hemostasis promoting method according to the present embodiment will generally be described below with reference to FIG. 4. The hemostasis assisting tool 10 is mounted on the forearm H1 (mounting step S1). The temperature of the body surface around (e.g., overlying) the radial artery V1 is made higher than the temperature of the body surface around (e.g., overlying) the ulnar artery V2 such that the temperature of the radial artery V1 will be higher than the temperature of the ulnar artery V2 (temperature adjusting step S2). The hemostasis assisting tool 10 is dismounted (dismounting step S3). The hemostasis promoting method according to the present embodiment will be described in detail below.

First, mounting step S1 will be described below.

As illustrated in FIGS. 1 and 2, the user such as a doctor or a nurse mounts the mount portion 20 on the forearm H1 of the patient such that the heating portion 31 is disposed on the heated region A1 and the cooling portion 32 is disposed on the cooled region A2.

Mounting step S1 may be carried out prior to or during the hemostasis. The hemostatic process is not limited to any particular process as long as it is capable of stanching the puncture site P. For example, a process of compressing the puncture site P with a known hemostatic device having a compression pad or the like may be used.

Next, temperature adjusting step S2 will be described below.

The heating portion 31 heats the body surface around the radial artery V1, and the cooling portion 32 cools the body surface around the ulnar artery V2. In this case, the temperature of the radial artery V1 increases, and the temperature of the ulnar artery V2 decreases. As a result, the radial artery V1 is expanded, and the ulnar artery V2 is contracted. The radial artery V1 and the ulnar artery V2 are blood vessels that branch from the brachial artery V0 and extend parallel to each other. Therefore, the volume of a blood flow in the radial artery V1 is increased as a part of the radial artery V1 being expanded, and the ulnar artery V2 is contracted. As a result, the amount of a blood coagulation factor such as platelets required to coagulate the blood in the radial artery V1 is increased. Therefore, the hemostasis on the puncture site P can be promoted. Further, the expansion of the radial artery V1 causes a reduction in the local blood pressure in the radial artery V1. Consequently, in a case where compression hemostasis is carried out, the compression force applied to the puncture site P is reduced by the reduction in the local blood pressure. Therefore, pain and numbness that the patient might otherwise feel by the compression hemostasis are restrained.

While the heating portion 31 is heating the body surface around the radial artery V1, the cooling portion 32 need not cool the body surface around the ulnar artery V2 (i.e., only the heating portion 31 may function). In this case, the temperature of the radial artery V1 rises. As a result, the radial artery V1 is expanded. Therefore, the volume of a blood flow in the radial artery V1 is increased as a part of the radial artery V1 being expanded. As a result, the amount of a blood coagulation factor in the radial artery V1 is increased. Therefore, the hemostasis on the puncture site P is promoted. Further, the expansion of the radial artery V1 causes a reduction in the local blood pressure in the radial artery V1. Consequently, in a case where compression hemostasis has been carried out, the compression force applied to the puncture site P is reduced by the reduction in the local blood pressure. Therefore, pain and numbness that the patient might otherwise feel by the compression hemostasis are restrained.

Further, while the cooling portion 32 is cooling the body surface around the ulnar artery V2, the heating portion 31 may not heat the body surface around the radial artery V1 (i.e., only the cooling portion 32 may function). In this case, the temperature of the ulnar artery V2 drops. As a result, the ulnar artery V2 is contracted. Therefore, the volume of blood flow in the radial artery V1 is increased as much as the ulnar artery V2 is contracted. As a result, the amount of a blood coagulation factor in the radial artery V1 is increased. Therefore, hemostasis on the puncture site P is promoted.

In this manner, hemostasis can be promoted by making the temperature of the radial artery V1 higher than the temperature of the ulnar artery V2 by using at least one of the heating portion 31 and the cooling portion 32 of the temperature adjusting portion 30.

If the user judges that the change in the amount of the blood flow is not sufficient in a case where either one of the heating portion 31 and the cooling portion 32 is used or operated, then the user may switch to functioning both the heating portion 31 and the cooling portion 32. Further, if the user judges that the change in the amount of the blood flow is not sufficient in a case where the amount of heating by the heating portion 31 and the amount of cooling by the cooling portion 32 are adjustable, then, the user may adjust the amount of heating by the heating portion 31 and/or the amount of cooling by the cooling portion 32 depending on the change in the amount of the blood flow. The change in the blood flow can be measured by ultrasonic echo, a laser rheometer, or the like, for example.

In the hemostasis promoting method according to the present embodiment, further, a portion of the radial artery V1 that is more to the center than (upstream of) the puncture site P is heated. That is, the portion that is heated is positioned more to an upstream side of the radial artery 1 (heart side) than the puncture site. The blood coagulation factor can thus be sent to the puncture site P after its temperature has been raised. Consequently, a biochemical reaction for coagulating the blood can be promoted.

Next, dismounting step S3 will be described below.

The user can dismount the hemostasis assisting tool 10 from the arm at the timing of completion of the hemostasis or the like, for example. However, the timing to dismount the hemostasis assisting tool 10 is not limited to any particular timing and may be prior to completion of the hemostasis as long as the effect of promotion of the hemostasis is obtained.

The hemostasis promoting method according to the above embodiment as described above is a hemostasis promoting method for promoting hemostasis on the puncture site P formed in the radial artery V1 among the radial artery V1 and the ulnar artery V2 that are branched from the brachial artery V0 in the arm and that extend parallel to each other. According to the hemostasis promoting method, the temperature of a body surface around the radial artery V1 is made higher than the temperature of a body surface around the ulnar artery V2 such that the temperature of the radial artery V1 will be higher than the temperature of the ulnar artery V2. Therefore, the volume of a blood flow in the radial artery V1 is increased. As a result, the amount of a blood coagulation factor in the radial artery V1 is increased. Therefore, the hemostasis on the puncture site P can be promoted.

In the hemostasis promoting method according to the above embodiment, further, the body surface around the radial artery V1 is heated, or the body surface around the ulnar artery V2 is cooled. Therefore, the volume of a blood flow (the amount of a blood coagulation factor) in the radial artery V1 can be increased by expanding the radial artery V1 or contracting the ulnar artery V2.

In the hemostasis promoting method according to the above embodiment, further, the body surface around the radial artery V1 is heated, and the body surface around the ulnar artery V2 is cooled. Therefore, the volume of a blood flow (the amount of a blood coagulation factor) in the radial artery V1 can be increased as much as the radial artery V1 is expanded and the ulnar artery V2 is contracted.

In the hemostasis promoting method according to the above embodiment, further, the body surface around a portion of the radial artery V1 that is more to the center than the puncture site P is heated. The heating portion is more to a position of upstream side of blood vessel (heart side) than the puncture site. The blood coagulation factor can thus be sent to the puncture site P after its temperature has been raised. Consequently, a biochemical reaction for coagulating the blood at the puncture site P can be promoted.

Further, the hemostasis assisting tool 10 according to the above embodiment is a hemostasis assisting tool mounted on the arm for assisting in stanching the puncture site P formed in the radial artery V1 among the radial artery V1 and the ulnar artery V2 that are branched from the brachial artery V0 in the arm and that extend parallel to each other. The hemostasis assisting tool 10 has the temperature adjusting portion 30 for making the temperature of a body surface around the radial artery V1 higher than the temperature of a body surface around the ulnar artery V2 such that the temperature of the radial artery V1 will be higher than the temperature of the ulnar artery V2. Therefore, the hemostasis assisting tool 10 can increase the volume of a blood flow in the radial artery V1. As a result, the amount of a blood coagulation factor in the radial artery V1 is increased. Therefore, the hemostasis assisting tool 10 can promote the hemostasis on the puncture site P.

Further, the temperature adjusting portion 30 has the heating portion 31 for heating the body surface around the radial artery V1 and the cooling portion 32 for cooling the body surface around the ulnar artery V2. Therefore, the volume of a blood flow (the amount of a blood coagulation factor) in the radial artery V1 can be increased as much as the radial artery V1 is expanded and the ulnar artery V2 is contracted.

Further, the heating portion 31 is disposed on the body surface around a portion of the radial artery V1 that is more to the center than the puncture site P. The blood coagulation factor can thus be sent to the puncture site P after its temperature has been raised. Consequently, a biochemical reaction for coagulating the blood at the puncture site P can be promoted.

(Modifications of a Region where a Temperature Difference is Developed)

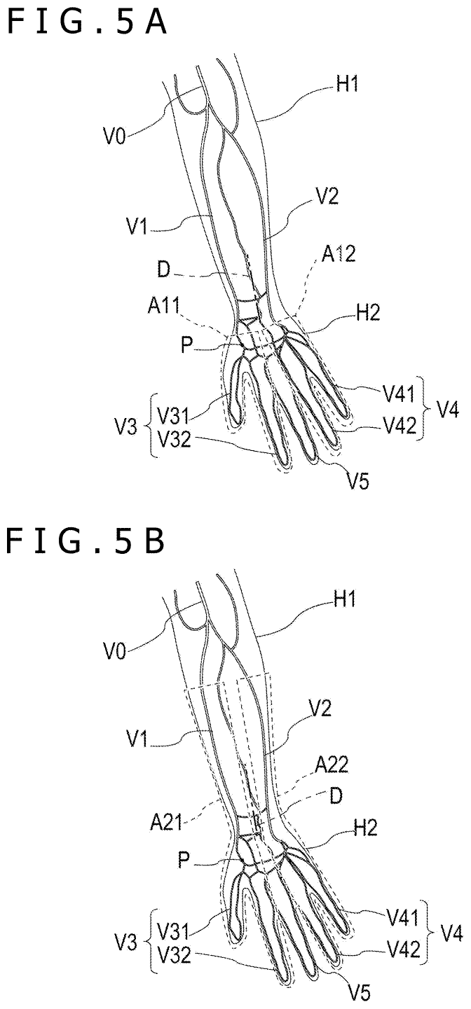

FIGS. 5A and 5B are views illustrating modifications of a region where a temperature difference is developed.

In a case where the puncture site P is formed in the radial artery V1, a region of the body surface where a temperature difference is to be developed is not limited to the regions A1 and A2 illustrated in FIG. 2.

As illustrated in FIG. 5A, for example, a region A11 of the body surface of the hand H2 that is on the thumb side of a portion extending from the palmaris longus muscle D to the middle finger as a boundary may be made higher in temperature than a region A12 on the little finger side. The temperature of a peripheral blood vessel V3 (corresponding to a peripheral blood vessel of the first blood vessel) of the radial artery V1 in the hand H2 can thus be made higher than the temperature of a peripheral blood vessel V4 (corresponding to a peripheral blood vessel of the second blood vessel) of the ulnar artery V2.

The phrase “peripheral blood vessel V3 of the radial artery V1” here means a blood vessel joined to the radial artery V1 and mainly supplied with blood from the radial artery V1 and refers, for example, to a proper palmar branch artery V31 of the thumb and a proper palmar branch artery V32 of the index finger. Further, the phrase “peripheral blood vessel V4 of the ulnar artery V2” here means a blood vessel joined to the ulnar artery V2 and mainly supplied with blood from the ulnar artery V2 and refers, for example, to a proper palmar branch artery V41 of the little finer and a proper palmar branch artery V42 of the ring finger.

Whether a proper palmar branch artery V5 of the middle finger is included in the peripheral blood vessel V3 of the radial artery V1 or the peripheral blood vessel V4 of the ulnar artery V2 depends on individual differences, etc. Therefore, a body surface around the proper palmar branch artery V5 of the middle finger may be a heated region for some patients or a cooled region for other patients or may be a buffer region that is not heated or cooled. In FIG. 5A, the body surface around the proper palmar branch artery V5 of the middle finger is illustrated as a buffer region.

The peripheral blood vessel V3 of the radial artery V1 in the hand H2 may thus be made higher than the temperature around the peripheral blood vessel V4 of the ulnar artery V2. In this case, the volume of a blood flow (the amount of a blood coagulation factor) in the radial artery V1 can be increased as a part of the peripheral blood vessel V3 of the radial artery V1 being expanded and/or the peripheral blood vessel V4 of the ulnar artery V2 being contracted. Consequently, the hemostasis on the puncture site P can be promoted.

Further, as illustrated in FIG. 5B, a region A21 of the body surface of the forearm H1 and hand H2 that is on the thumb side of a portion extending from the palmaris longus muscle D to the middle finger as a boundary may be made higher in temperature than a region A22 on the little finger side. In this case, the temperature of both the radial artery V1 and the peripheral blood vessel V3 of the radial artery V1 is made higher than the temperature of both the ulnar artery V2 and the peripheral blood vessel V4 of the ulnar artery V2. The volume of a blood flow (the amount of a blood coagulation factor) in the radial artery V1 can thus be increased to a great extent compared with the hemostasis promoting method according to the above embodiment.

(Modifications of a Heating Process and a Cooling Process)

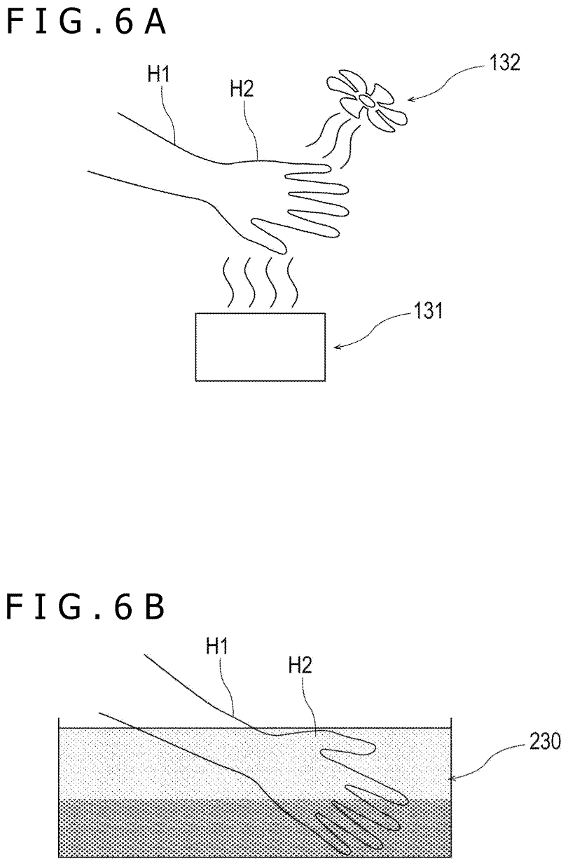

FIGS. 6A and 6B are views illustrating modifications of a heating process and a cooling process.

A heating process and a cooling process are not limited to the processes using the hemostasis assisting tool 10 according to the above embodiment.

As illustrated in FIG. 6A, for example, a body surface may be heated by a known heating instrument 131 using a heat transfer by way of conduction of air, steam, or the like or a heat transfer by way of heat radiation. In particular, in a case where the body surface on the thumb (radial artery) side is heated by the heating instrument 131 using steam, part of the steam may be applied to the body surface on the little finger (ulnar artery) side. Consequently, the body surface on the little finger (ulnar artery) side can be cooled by the heat of evaporation of the steam. Further, as illustrated in FIG. 6A, for example, the body temperature of the little finger side may further be cooled by a cooling instrument 132 such as an air blower.

Further, as illustrated in FIG. 6B, for example, a temperature difference may be developed by putting the hand H2 into a water container 230 in which water is not stirred and having a temperature distribution where the temperature is higher on the upper side than on the bottom side, such that the thumb is positioned on the upper side and the little finger on the bottom side.

Further, heating may be performed by a pouch of warm water or the like or by warm carbonated water, for example.

Further, cooling may be performed by a pouch of iced water or the like or by a cooling spray or the like, for example.

Further, in a case where heating and cooling are performed by a heating instrument and a cooling instrument using a heat transfer by way of convection, a partition may be placed between a heating region and a cooling region.

### Second Embodiment

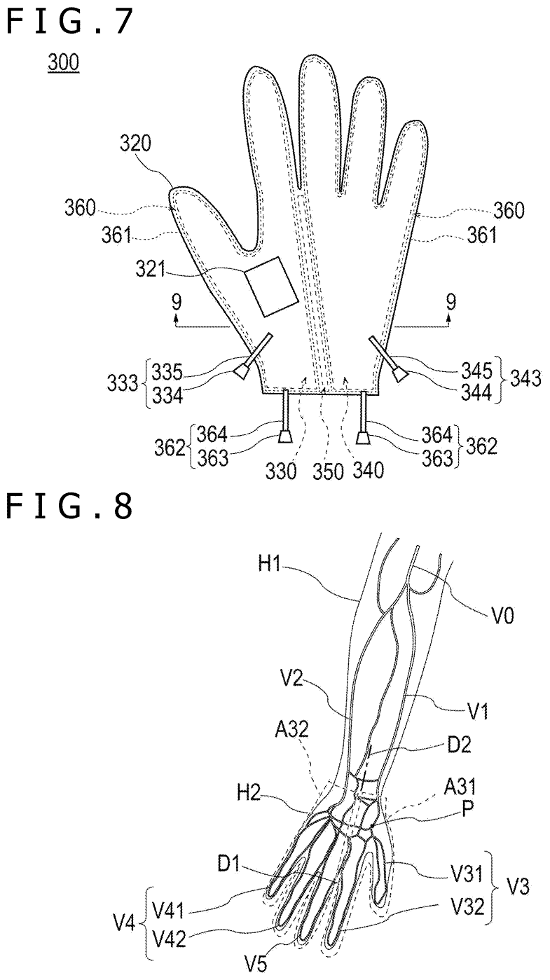

FIGS. 7 through 9 are views illustrating a hemostasis assisting tool 300 according to a second embodiment.

As illustrated in FIG. 8, the hemostasis assisting tool 300 according to the second embodiment is constructed as a device that assists in stanching the puncture site P formed in the radial artery V1 among the radial artery V1 and the ulnar artery V2 that are branched from the brachial artery V0 in the arm and that extend parallel to each other.

According to the second embodiment, the hand H2 will be described as a right hand, with the puncture site P formed in the d-RA of the patient's right hand. However, the puncture site P may be formed in a left hand.

The hemostasis assisting tool 300 according to the present embodiment will generally be described below with reference to FIGS. 7 and 8. The hemostasis assisting tool 300 has a mount portion 320 to be mounted on the hand H2, a heating portion 330 disposed on the mount portion 320, for heating a body surface around the radial artery V1 and the peripheral blood vessel V3 of the radial artery V1, a cooling portion 340 for cooling a body surface around the ulnar artery V2 and the peripheral blood vessel V4 of the ulnar artery V2, a heat transfer restraining portion 350 disposed between the heating portion 330 and the cooling portion 340, and covering portions 360 covering surfaces of the heating portion 330 and the cooling portion 340 that face the body surface.

As described above, the phrase “peripheral blood vessel V3 of the radial artery V1” here means a blood vessel joined to the radial artery V1 and mainly supplied with blood from the radial artery V1 and refers, for example, to the proper palmar branch artery V31 of the thumb and the proper palmar branch artery V32 of the index finger, as illustrated in FIG. 8. Further, the phrase “peripheral blood vessel V4 of the ulnar artery V2” here means a blood vessel joined to the ulnar artery V2 and mainly supplied with blood from the ulnar artery V2 and refers, for example, to the proper palmar branch artery V41 of the little finer and the proper palmar branch artery V42 of the ring finger.

Further, as described above, whether the proper palmar branch artery V5 of the middle finger is included in the peripheral blood vessel V3 of the radial artery V1 or the peripheral blood vessel V4 of the ulnar artery V2 depends on individual differences, etc. Therefore, the body surface around the proper palmar branch artery V5 of the middle finger may be a heated region A1 heated by the heating portion 330 for some patients or a cooled region A2 cooled by the cooling portion 340 for other patients or may be a buffer region that is not heated or cooled. According to the present embodiment, the body surface around the proper palmar branch artery V5 of the middle finger is illustrated as being included in the cooled region A2. Parts of the hemostasis assisting tool 300 will hereinafter be described in detail.

(Mount Portion)

According to the present embodiment, as illustrated in FIG. 7, the mount portion 320 is constructed as a glove. In FIG. 7, the mount portion 320 is illustrated as a five-finger-type glove including parts where five fingers are separately insertable and which cover the five fingers up to their fingertips. However, the mount portion 320 may be a mitten-type glove or a five-finger-type glove from which the fingertips are exposed.

As illustrated in FIG. 7, the mount portion 320 has an opening 321 where the puncture site P (see FIG. 8) formed in the d-RA of the hand H2 can be exposed. Therefore, a compressive hemostatic device including a compression pad or the like can be pressed against the puncture site P exposed from the hemostasis assisting tool 300. The position of the opening 321 can appropriately be changed in design depending on the position of the puncture site P. For example, in a case where a puncture site is formed in the s-RA of the hand H2, the opening 321 is formed in a position on the mount portion 320 where the s-RA can be exposed.

The material of the mount portion 320 is not limited to any particular material as long as it has flexibility and may be polyester, nylon, or the like, for example.

(Heating Portion)

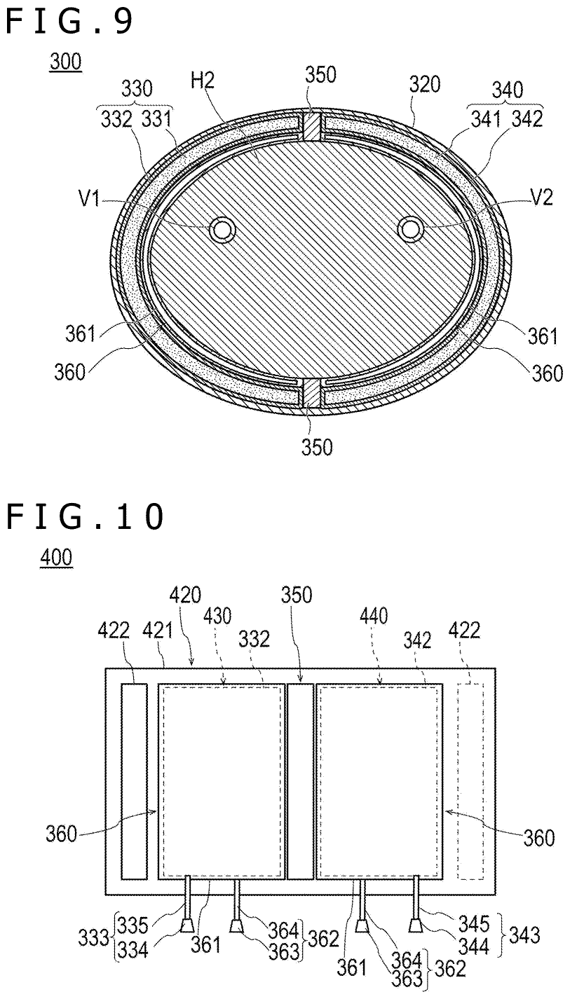

As illustrated in FIGS. 7 and 9, the heating portion 330 has a first housing portion 332 housing therein a heat generating substance 331 that generates heat by reacting with a fluid (not illustrated) and a first injection starting mechanism (first injection mechanism) 333 for starting injection of (injecting) the fluid into the first housing portion 332. Parts of the heating portion 330 will hereinafter be described in detail.

According to the present embodiment, the first housing portion 332 is constructed as a flexible pouch. However, the configuration of the first housing portion 332 is not limited to any particular configuration as long as it can house the heat generating substance 331 therein.

The fluid that reacts with the heat generating substance 331 may be either a liquid or a gas, but should preferably be a liquid, more preferably an aqueous solution, and much more preferably water.

The heat generating substance 331 is an inorganic oxide or an inorganic hydroxide. The inorganic oxide may be, for example, an oxide alkali metal salt such as sodium oxide or potassium oxide, an oxide alkali earth metal salt such as calcium oxide or magnesium oxide, or the like. The inorganic hydroxide may be, for example, a hydroxide alkali metal salt such as sodium hydroxide or potassium hydroxide, a hydroxide alkali earth metal salt such as calcium hydroxide or magnesium hydroxide, or the like. Of these materials, calcium oxide (quicklime) is preferable as the heat generating substance 331 from the standpoint of heat generating capability.

The form of the heat generating substance 331 is not limited to any particular form, but should preferably be of a powdery (powder) form from the standpoint of safety. The heat generating substance 331 may be of one kind alone or two or more kinds in combination.

A heat generation assistant may be added to the heat generating substance 331. The heat generation assistant may be any of metals; inorganic chlorides; inorganic sulfides; acid substances (for example, organic acids, inorganic acids, their salts, or the like); glycols; polyglycols, or the like. Of these materials, metals are preferable, aluminum or zinc is more preferable, or aluminum is much more preferable as the heat generation assistant because their heat generation can easily be controlled.

According to the present embodiment, as illustrated in FIG. 7, the first injection starting mechanism 333 has a check valve 334 for allowing the fluid to be injected with a known injection instrument (not illustrated) such as a syringe and preventing the injected fluid from leaking out; and a tube 335 that provides fluid communication between the check valve 334 and the first housing portion 332. When the injection instrument has its distal portion inserted into the check valve 334, the check valve 334 is opened, starting injection of the fluid into the first housing portion 332.

However, the configuration of the first injection starting mechanism 333 is not limited to any particular configuration as long as it can start injection of the fluid into the first housing portion 332. For example, the first injection starting mechanism 333 may be constructed as a pouch of thin film that houses the fluid therein and that is ruptured when a pressure equal to or higher than a reference value is applied thereto. In this case, when crushed, the first injection starting mechanism 333 is ruptured, starting injection of the fluid into the first housing portion 332.

According to the present embodiment, as illustrated in FIG. 9, the heating portion 330 is disposed to an inner surface (a surface that faces the body surface in a mounted state) of the mount portion 320. However, the heating portion 330 may be disposed on an outer surface (a surface that faces outwardly in a mounted state) of the mount portion 320. In a case where the mount portion 320 has a multilayer structure, the heating portion 330 may be disposed between layers of the mount portion 320.

According to the present embodiment, as illustrated in FIG. 8, a heated region A31 of the body surface that is heated by the heating portion 330 is a region that is on the thumb side of a portion extending from a point D1 between the middle finger and the index finger to the palmaris longus muscle D2 as a boundary. However, the heated region A31 is not limited to any particular region as long as it includes a body surface around (e.g., overlying) the radial artery V1 and/or the peripheral blood vessel V3 of the radial artery V1. The phrase “body surface around the radial artery V1 and/or the peripheral blood vessel V3 of the radial artery V1” here means a region of the body surface which, when heated by the heating portion 31, can transmit heat to the radial artery V1 and/or the peripheral blood vessel V3 of the radial artery V1 to increase the temperature of the radial artery V1 and/or the peripheral blood vessel V3 of the radial artery V1. Therefore, the heated region A31 may be, for example, only a region of the body surface of the hand H2 that is positioned directly above the radial artery V1 when the hand H2 is viewed in plan from the palmar side (or from the back of the hand H2). Alternatively, the heated region A31 may be only the thumb or the index finger.

According to the present embodiment, the heating portion 330 is disposed in surrounding relation to the opening 321. Therefore, the heating portion 330 can heat a body surface near the puncture site P.

The heating portion 330 should, in a mounted state, preferably be able to increase the temperature of the body surface to a temperature higher than the average body surface temperature around at least the puncture site P of the patient (for example, the average of body temperatures measured for several minutes at the hand H2, the arm, the armpit, etc., prior to the heating). The heating portion 330 should preferably, but not necessarily, be able to increase the temperature of the body surface up to a range of 33° C. to 37° C.

(Cooling Portion)

As illustrated in FIGS. 7 and 9, the cooling portion 340 has a second housing portion 342 housing therein a heat absorbing substance 341 that absorbs heat by reacting with a fluid (not illustrated) and a second injection starting mechanism (second injection mechanism) 343 for starting injection of (injecting) the fluid into the second housing portion 342. Parts of the cooling portion 340 will hereinafter be described in detail.

According to the present embodiment, the second housing portion 342 is constructed as a flexible pouch. However, the configuration of the second housing portion 342 is not limited to any particular configuration as long as it can house the heat absorbing substance 341 therein.

The fluid that reacts with the heat absorbing substance 341 may be either a liquid or a gas, but should preferably be a liquid, more preferably an aqueous solution, and much more preferably water. The fluid that reacts with the heat absorbing substance 341 and the fluid that reacts with the heat generating substance 331 may be the same fluid or may be different fluids.

The heat absorbing substance 341 may be an ammonium salt such as ammonium chloride, ammonium nitrate, or ammonium sulfate; a sodium salt hydrate such as sodium carbonate, sodium sulfate hydrate, sodium carbonate hydrate, or sodium hydrogensulfate; urea; sugar alcohol such as monosaccharide alcohol such as xylitol, erythritol, or sorbitol, or the like. The heat absorbing substance may be of one kind alone or two or more kinds in combination. Of these materials, an ammonium salt or urea is preferable or ammonium sulfate or urea is more preferable as the heat absorbing substance 341 from the standpoint of heat absorbing capability.

The form of the heat absorbing substance 341 is not limited to any particular form, but should preferably be of a powdery (powder) form from the standpoint of stability. The heat absorbing substance 341 may be of one kind alone or two or more kinds in combination.

According to the present embodiment, as illustrated in FIG. 7, the second injection starting mechanism 343 has a check valve 344 for allowing the fluid to be injected with a known injection instrument (not illustrated) such as a syringe and preventing the injected fluid from leaking out; and a tube 345 that provides fluid communication between the check valve 344 and the second housing portion 342. When the injection instrument has its distal portion inserted into the check valve 344 and injects the fluid, the check valve 344 starts injection of the fluid into the second housing portion 342.

However, the configuration of the second injection starting mechanism 343 is not limited to any particular configuration as long as it can start injection of the fluid into the second housing portion 342. For example, the second injection starting mechanism 343 may be constructed as a pouch of thin film that houses the fluid therein and that is ruptured when a pressure equal to or higher than a reference value is applied thereto. In this case, when crushed, the second injection starting mechanism 343 is ruptured, starting injection of the fluid into the second housing portion 342.

According to the present embodiment, as illustrated in FIG. 9, the cooling portion 340 is disposed to an inner surface of the mount portion 320. However, the cooling portion 340 may be disposed on an outer surface (a surface that faces outwardly in a mounted state) of the mount portion 320. In a case where the mount portion 320 has a multilayer structure, the cooling portion 340 may be disposed between layers of the mount portion 320.

According to the present embodiment, as illustrated in FIG. 8, a cooled region A32 of the body surface that is heated by the cooling portion 340 is a region that is on the little finger side of a portion extending from the point D1 between the middle finger and the index finger to the palmaris longus muscle D2 as a boundary. However, the cooled region A32 is not limited to any particular region as long as it includes a body surface around (e.g., overlying) the ulnar artery V2 and/or the peripheral blood vessel V4 of the ulnar artery V2. The phrase “body surface around the ulnar artery V2 and/or the peripheral blood vessel V4 of the ulnar artery V2” here means a region of the body surface which can transmit heat from the ulnar artery V2 and/or the peripheral blood vessel V4 of the ulnar artery V2 to the cooling portion 40 to reduce the temperature of the ulnar artery V2 and/or the peripheral blood vessel V4 of the ulnar artery V2. Therefore, the cooled region A32 may be, for example, only a region of the body surface of the hand H2 that is positioned directly above the ulnar artery V2 when the hand H2 is viewed in plan from the palmar side (or from the back of the hand H2). Alternatively, the cooled region A32 may be only the little finger or the ring finger.

The cooling portion 340 should, in a mounted state, preferably be able to reduce the temperature of the body surface to a temperature lower than at least the average body surface temperature of the patient (for example, the average of body temperatures measured for several minutes at the hand H2, the arm, the armpit, etc.). The cooling portion 340 should preferably, but not necessarily, be able to reduce the temperature of the body surface down to a range of 10° C. to 27° C.

(Heat Transfer Restraining Portion)

As illustrated in FIGS. 7 and 9, the heat transfer restraining portion 350 is attached to the mount portion 320 while being disposed between the heating portion 330 and the cooling portion 340.

The heat transfer restraining portion 350 is not limited to any particular form as long as it can restrain heat from being transferred between the heating portion 330 and the cooling portion 340, and may be constructed as a pouch filled with a fibrous heat insulator such as glass wool, a foamed heat insulator such as urethane foam, or a gas such as air, or as a gap, a partition, or the like disposed between the heating portion 330 and the cooling portion 340. The hemostasis assisting tool 300 may be free of the heat transfer restraining portion 350.

(Covering Portions)

As illustrated in FIG. 9, the covering portions 360, in a mounted state, are disposed between the heating portion 330 and the cooling portion 340 and the body surface. Therefore, the covering portions 360 restrain an excessive heat transfer between the heating portion 330 and the cooling portion 340 and the hand H2 while allowing the body surface to be heated by the heating portion 330 and also allowing the body surface to be cooled by the cooling portion 340. In the present description, the phrase “excessive heat transfer” means a heat transfer intensive enough to cause the hand to be burned by being heated by the heating portion 330 or to be frostbitten by being cooled by the cooling portion 340.

According to the present embodiment, as illustrated in FIG. 9, the covering portions 360 include two covering portions 360 disposed individually on the inner surface of the heating portion 330 and the inner surface of the cooling portion 340.

According to the present embodiment, as illustrated in FIGS. 7 and 9, each of the covering portions 360 has a flexible pouch 361 that can be filled with a gas and an injection port 362 coupled to the pouch 361. The injection port 362 has a check valve 363 for allowing a gas such as air to be injected with a known injection instrument such as a syringe and preventing the injected gas from leaking out; and a tube 364 that provides fluid communication between the check valve 363 and the pouch 361. The covering portions 360 have a thickness that varies depending on the amount of the gas injected through the injection ports 362. Therefore, the covering portions 360 are capable of adjusting the extent to which the heat transfer is restrained, by adjusting the amount of the gas injected through the injection ports 362 (the thickness of the covering portions 360).

The configuration of the covering portions 360 is not limited to any particular configuration as long as it can restrain an excessive heat transfer between the heating portion 330 and the cooling portion 340 and the hand H2. The covering portions 360 may be made of a sheet material, for example. The hemostasis assisting tool 300 may be free of the covering portions 360, providing the maximum temperature of the heating portion 330 and the minimum temperature of the cooling portion 340 are set to such temperatures as not to cause frostbiting and burning.

(Method of Use)

Next, a method of using the hemostasis assisting tool 300 according to the present embodiment will be described in detail below with respect to an example where the puncture site P formed in the d-RA of the hand H2 is to be stanched.

First, the user such as a doctor or a nurse mounts the hemostasis assisting tool 300 on the hand H2. Consequently, as illustrated in FIG. 8, the heating portion 330 is disposed on the heated region A31, and the cooling portion 340 is disposed on the cooled region A32.

The hemostasis assisting tool 300 may be mounted prior to or during the hemostasis. The hemostatic process is not limited to any particular process as long as it is capable of stanching the puncture site P. For example, a process of compressing the puncture site P with a known hemostatic device having a compression pad or the like may be used.

Next, the user such as a doctor or a nurse injects a fluid into the heating portion 330 and the cooling portion 340 using a known injection instrument such as a syringe. Further, the user such as a doctor or a nurse injects a gas such as air into the covering portions 360 using a known injection instrument such as a syringe.

Consequently, the heating portion 330 heats the heated region A31, and the cooling portion 340 cools the cooled region A32.

Therefore, the temperature of the radial artery V1 and the peripheral blood vessel V3 of the radial artery V1 rises, and the temperature of the ulnar artery V2 and the peripheral blood vessel V4 of the ulnar artery V2 drops. As a consequence, the radial artery V1 and the peripheral blood vessel V3 of the radial artery V1 are expanded, and the ulnar artery V2 and the peripheral blood vessel V4 of the ulnar artery V2 are contracted. The radial artery V1 and the ulnar artery V2 branch from the brachial artery V0 and extend parallel to each other. Therefore, the volume of a blood flow in the radial artery V1 is increased as a part of the radial artery V1 and the peripheral blood vessel V3 of the radial artery V1 being expanded and the ulnar artery V2 and the peripheral blood vessel V4 of the ulnar artery V2 being contracted. As a result, the amount of a blood coagulation factor such as platelets required to coagulate the blood in the radial artery V1 is increased. Therefore, the hemostasis on the puncture site P can be promoted.

Further, the expansion of the radial artery V1 causes a reduction in the local blood pressure in the radial artery V1. Consequently, in a case where compression hemostasis is carried out, the compression force applied to the puncture site P is reduced by the reduction in the local blood pressure. Therefore, pain and numbness that the patient might otherwise feel by the compression hemostasis are restrained.

In a case where the change in the amount of the blood flow is not sufficient, the user such as a doctor or a nurse may adjust the amount of the fluid injected into the heating portion 330 and the cooling portion 340 and/or the amount of the gas injected into the covering portions 360. The change in the blood flow can be measured by ultrasonic echo, a laser rheometer, or the like, for example.

The heating and cooling may be started during or prior to the hemostasis.

Next, the user such as a doctor or a nurse can dismount the hemostasis assisting tool 300 from the hand H2 at the time of completion of the hemostasis or the like, for example. However, the timing to dismount the hemostasis assisting tool 300 is not limited to any particular timing, and may be prior to completion of the hemostasis as long as the effect of promotion of the hemostasis is obtained.

The hemostasis assisting tool 300 according to the second embodiment as described above is a hemostasis assisting tool mounted on the hand H2 for assisting in stanching the puncture site P formed in the radial artery V1 among the radial artery V1 and the ulnar artery V2 that are branched from the brachial artery V0 in the arm and that extend parallel to each other. The hemostasis assisting tool 300 has the heating portion 330 for heating the body surface around the radial artery V1 and the peripheral blood vessel V3 of the radial artery V1 and the cooling portion 340 for cooling the body surface around the ulnar artery V2 and the peripheral blood vessel V4 of the ulnar artery V2 while being mounted on the hand H2.

With the above hemostasis assisting tool 300, the heating portion 330 heats the radial artery V1 and the peripheral blood vessel V3 of the radial artery expanding the radial artery V1 and the peripheral blood vessel V3 of the radial artery V1. In addition, the cooling portion 340 cools the ulnar artery V2 and the peripheral blood vessel V4 of the ulnar artery V2, contracting the ulnar artery V2 and the peripheral blood vessel V4 of the ulnar artery V2. The volume of a blood flow in the radial artery V1 is increased as a part of the radial artery V1 and the peripheral blood vessel V3 of the radial artery V1 being expanded and the ulnar artery V2 and the peripheral blood vessel V4 of the ulnar artery V2 being contracted. As a result, the amount of a blood coagulation factor in the radial artery V1 is increased. Therefore, the hemostasis on the puncture site P formed in the radial artery V1 can be promoted.

Further, the heating portion 330 has the first housing portion 332 housing therein the heat generating substance 331 that generates heat by reacting with a fluid and the first injection starting mechanism 333 for starting injection of the fluid into the first housing portion 332. The cooling portion 340 has the second housing portion 342 housing therein the heat absorbing substance 341 that absorbs heat by reacting with a fluid and the second injection starting mechanism 343 for starting injection of the fluid into the second housing portion 342. Since heating and cooling can be performed by a chemical reaction, the heating portion 330 and the cooling portion 340 can be made simple in configuration compared with a case where heating and cooling are electrically performed.

Further, the heat transfer restraining portion 350 capable of restraining a heat transfer between the heating portion 330 and the cooling portion 340 is disposed between the heating portion 330 and the cooling portion 340. Therefore, a heat transfer between the heating portion 330 and the cooling portion 340 can be restrained.

Further, the hemostasis assisting tool 300 has the covering portions 360 that cover surfaces of the heating portion 330 and the cooling portion 340 that face the body surface. Consequently, the covering portions 360 can restrain the heating portion 330 and the cooling portion 340 from directly contacting the body surface, thus restraining the body surface from being burned and frostbitten due to an excessive heat transfer.

Further, each of the covering portions 360 has the pouch 361 that can be filled with a gas and the injection port 362 that allows a gas to be injected with the injection instrument into the pouch 361 and prevents the injected gas from leaking out. Therefore, the thickness of the covering portions 360 can be adjusted by adjusting the amount of the gas injected into the pouch 361, thereby adjusting the extent of a heat transfer between the heating portion 330 and the cooling portion 340 and the hand H2.

Further, the hemostasis assisting tool 300 has the opening 321 where the puncture site P can be exposed. Therefore, the hemostasis assisting tool 300 can be used while the hemostatic device is being mounted on the hand H2.

### Third Embodiment

FIGS. 10 through 12 are views illustrating a hemostasis assisting tool 400 according to a third embodiment.

The hemostasis assisting tool 400 according to the third embodiment is different from the hemostasis assisting tool 300 according to the second embodiment in that the hemostasis assisting tool 400 is mounted on the forearm H1, instead of the hand H2. The hemostasis assisting tool 400 according to the third embodiment will hereinafter be described in detail.

The hemostasis assisting tool 400 will generally be described below with reference to FIG. 10. The hemostasis assisting tool 400 has a mount portion 420, a heating portion 430, a cooling portion 440, a heat transfer restraining portion 350, and covering portions 360. Those parts which are identical to those of the hemostasis assisting tool 300 according to the second embodiment are denoted by identical reference symbols and a detailed description of such features will not be repeated.

(Mount Portion)

According to the present embodiment, as illustrated in FIG. 10, the mount portion 420 has a band body (band) 421 that can be wound around the arm and a joint member 422 for joining the ends of the band body 421 to each other. Parts of the mount portion 420 will hereinafter be described.

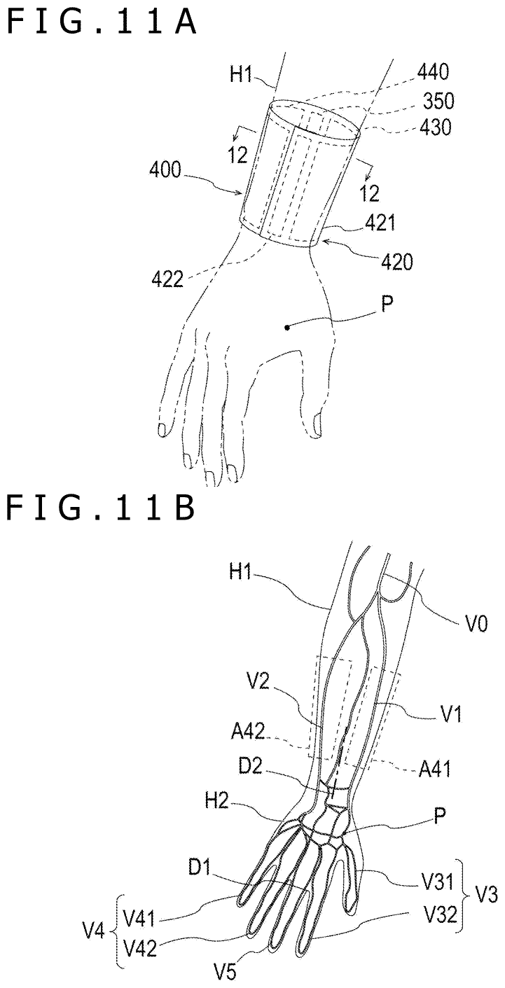

According to the present embodiment, the band body 421 is constructed as one or two or more flexible sheet members. As illustrated in FIG. 11A, the band body 421 is wound substantially fully around the forearm H1.

The material from which the band body 421 may be fabricated is not limited to any particular material as long as it has flexibility, and may be polyester, nylon, or the like, for example.

The joint member 422 is not limited to any particular joint member as long as it can join the ends of the band body 421 to each other while the band body 421 is wound around the forearm H1. The joint member 422 may, for example, be a surface fastener generally called Magic Tape (registered trademark), a snap, a button, a clip, a frame member through which an end of the band body 421 passes, or the like. In FIG. 10, the joint member 422 is illustrated as having a male part (or a female part) of a surface fastener disposed on one end of the band body 421 and a female part (or a male part) of the surface fastener on the other end of the band body 421. As illustrated in FIG. 11A, the hemostasis assisting tool 400 is mounted on the forearm H1 when the ends of the band body 421 are joined to each other by the joint member 422 while the band body 421 is wound around the forearm H1.

The configuration of the mount portion 420 is not limited to any particular configuration as long as it can be mounted on the forearm H1. The mount portion 420 may, for example, be an annular member through which the forearm H1 can be inserted, instead of the band body 421.

(Heating Portion)

According to the present embodiment, the heating portion 430 heats a body surface of the forearm H1 around the radial artery V1 (see FIG. 11B).

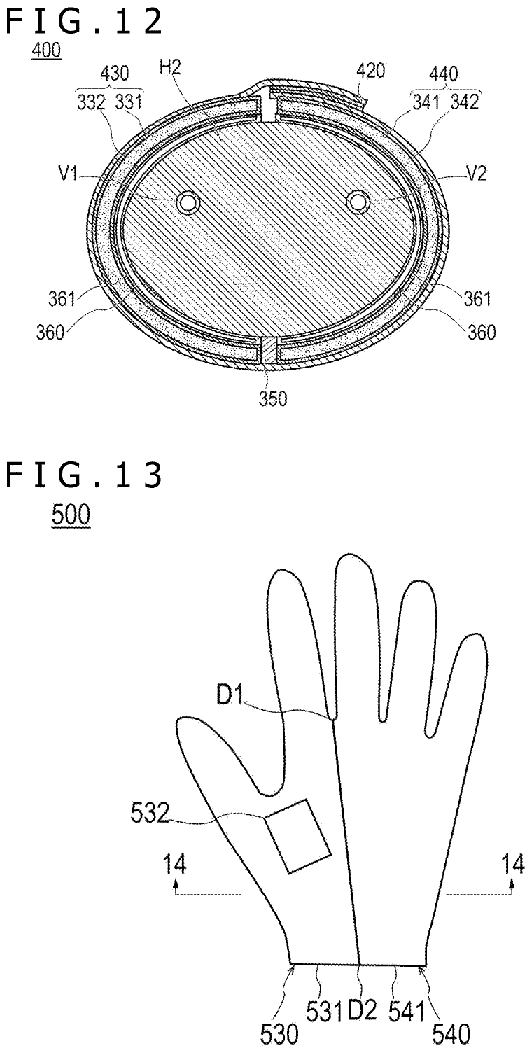

According to the present embodiment, as illustrated in FIG. 12, the heating portion 430 is disposed on an inner surface of the mount portion 420. However, the heating portion 430 may be disposed on an outer surface (a surface that faces outwardly in a mounted state) of the mount portion 420. In a case where the mount portion 420 has a multilayer structure, the heating portion 430 may be disposed between layers of the mount portion 420.

According to the present embodiment, as illustrated in FIG. 11B, a heated region A41 of the body surface that is heated by the heating portion 430 is a region of the body surface of the forearm H1 that is on the thumb side of the palmaris longus muscle D2 as a boundary. However, the heated region A41 is not limited to any particular region as long as it includes the body surface around the radial artery V1. The heated region A41 may be, for example, only a region of the body surface of the forearm H1 that is positioned directly above the radial artery V1 when the forearm H1 is viewed in plan from the palmar side (or from the back of the hand H2).

(Cooling Portion)

The cooling portion 440 cools a body surface of the forearm H1 around the ulnar artery V2 (see FIG. 11B).

According to the present embodiment, as illustrated in FIG. 12, the cooling portion 440 is disposed on an inner surface of the mount portion 420. However, the cooling portion 440 may be disposed on an outer surface (a surface that faces outwardly in a mounted state) of the mount portion 420. In a case where the mount portion 420 has a multilayer structure, the cooling portion 440 may be disposed between layers of the mount portion 420.

According to the present embodiment, as illustrated in FIGS. 11A and 11B, a cooled region A42 of the body surface that is cooled by the cooling portion 440 is a region of the body surface of the forearm H1 that is on the little finger side of the palmaris longus muscle D2 as a boundary. However, the cooled region A42 is not limited to any particular region as long as it includes the body surface around the ulnar artery V2. The cooled region A42 may be, for example, only a region of the body surface of the forearm H1 that is positioned directly above the ulnar artery V2 when the forearm H2 is viewed in plan from the palmar side (or from the back of the hand H2).

With the hemostasis assisting tool 400 according to the third embodiment, as described above, the heating portion 430 is disposed on the body surface around a portion of the radial artery V1 that is more to the center than the puncture site P. The blood coagulation factor can thus be sent to the puncture site P after it has been heated by the heating portion 430. Consequently, a biochemical reaction for coagulating the blood at the puncture site P can be promoted.

### Fourth Embodiment

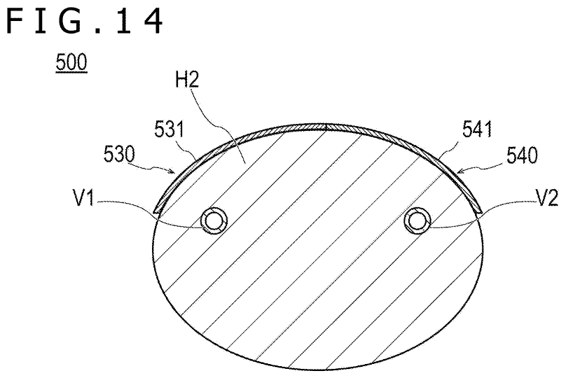

FIGS. 13 and 14 are views illustrating a hemostasis assisting tool 500 according to a fourth embodiment.

The hemostasis assisting tool 500 according to the fourth embodiment is different from the hemostasis assisting tool 300 according to the second embodiment in that the hemostasis assisting tool 500 is free of the mount portion 320 and the heat transfer restraining portion 350 and the configurations of the heating portion 530 and the cooling portion 540 are different. The hemostasis assisting tool 500 according to the fourth embodiment will hereinafter be described.

The hemostasis assisting tool 500 has a heating portion 530 and a cooling portion 540. Note that those parts which are identical to those of the hemostasis assisting tool 300 according to the second embodiment are denoted by identical reference symbols and will not be described below.

(Heating Portion)

According the present embodiment, the heating portion 530 heats the radial artery V1 and the peripheral blood vessel V3 of the radial artery V1 (see FIG. 8).

The heating portion 530 is constructed as a heating sheet 531 affixed to a body surface around the radial artery V1 and the peripheral blood vessel V3 of the radial artery V1, for heating the body surface.

As illustrated in FIGS. 13 and 14, the heating sheet 531 is arranged to cover a region on the back side on the thumb side of a portion extending from a point D1 between the middle finger and the index finger to the palmaris longus muscle D2 as a boundary. However, the heated region is not limited to any particular region as long as it includes a body surface around (e.g., overlying) the radial artery V1 and/or the peripheral blood vessel V3 of the radial artery V1. The heated region may, for example, be a region on the palmar side.

As illustrated in FIG. 13, the heating sheet 531 has an opening 532 where the puncture site P formed in the d-RA of the hand H2 can be exposed. Therefore, a publicly known compressive hemostatic device including a compression pad or the like can be pressed against the puncture site P exposed from the hemostasis assisting tool 500 The position of the opening 532 can appropriately be changed in design depending on the position of the puncture site P. For example, in a case where a puncture site is formed in the s-RA of the hand H2, the opening 532 is formed in a portion of the heating sheet 531 where a region around the s-RA can be exposed.

The heating sheet 531 contains a heating component such as capsicum extract or nonylic acid vanillylamide and a known adhesive component such as an acrylic adhesive, a silicone adhesive, a polyvinyl adhesive, a polyester adhesive, or a polyurethane adhesive that can adhere to the body surface.

(Cooling Portion)

The cooling portion 540 cools the ulnar artery V2 and the peripheral blood vessel V4 of the ulnar artery V2 (see FIG. 8).

As illustrated in FIGS. 13 and 14, the cooling portion 540 is constructed as a cooling sheet 541 affixed to a body surface around the ulnar artery V2 and the peripheral blood vessel V4 of the ulnar artery V2, for cooling the body surface.

As illustrated in FIG. 13, the cooling sheet 541 is arranged to cover a region on the back side on the little finger side of a portion extending from the point D1 between the middle finger and the index finger to the palmaris longus muscle D2 as a boundary. However, the cooled region is not limited to any particular region as long as it includes a body surface around the ulnar artery V2 and the peripheral blood vessel V4 of the ulnar artery V2. The cooled region may, for example, be a region on the palmar side.

The cooling sheet 541 contains a cooling component such as methyl salicylate, menthol, or mint oil and a known adhesive component such as an acrylic adhesive, a silicone adhesive, a polyvinyl adhesive, a polyester adhesive, or a polyurethane adhesive that can adhere to the body surface.

The heating sheet 531 and the cooling sheet 541 are joined to each other by a known process such as adhesive bonding or fusion. A heat exchange restraining portion for restraining a heat exchange between the heating sheet 531 and the cooling sheet 541 may be disposed between the heating sheet 531 and the cooling sheet 541.

Protective sheets (not illustrated) are affixed to inner surfaces (adhesive surfaces) of the heating sheet 531 and the cooling sheet 541 at least at a time prior to use. By peeling off the protective sheets from the heating sheet 531 and the cooling sheet 541, it is possible to start heating peripheral blood vessels V11 and 12 of the radial artery V1 and peripheral blood vessels V21 and 22 of the ulnar artery V2.

With the hemostasis assisting tool 500 according to the fourth embodiment, as described above, the heating portion 530 has the heating sheet 531 affixed to the body surface around the radial artery V1 and the peripheral blood vessel V3 of the radial artery V1, for heating the body surface. Further, the cooling portion 540 has the cooling sheet 541 affixed to the body surface around the ulnar artery V2 and the peripheral blood vessel V4 of the ulnar artery V2, for cooling the body surface. Therefore, the user can apply the hemostasis assisting tool 500 to a limb such as the hand H2 by taking a simple action of affixing the hemostasis assisting tool 500 to a body surface thereof.

Although the promoting methods and the hemostasis assisting tools according to the embodiments and modifications have been described above, the hemostasis promoting method and the hemostasis assisting tool according to the present invention are not limited to the arrangements described in the embodiments and modifications, and may appropriately be altered on the basis of the scope of the claims for patent that are described.

For example, though the puncture site has been illustrated as being formed in the radial artery in the embodiments and modifications, a puncture site may be formed in the ulnar artery. In such a case, the temperature of the body surface around the ulnar artery and/or the peripheral blood vessel of the ulnar artery may be made higher than the temperature of the body surface around the radial artery and/or the peripheral blood vessel of the radial artery.

Alternatively, on a leg, when a puncture site is formed at an artery such as a plantar artery of the foot, a dorsal artery of the foot, a peroneal artery, an anterior tibial artery and a posterior tibial artery, the temperature of the body surface around the artery at which the puncture site is located may be made higher than the temperature of a body surface around the peripheral blood vessel of either one of the plantar artery and/or the dorsal artery of the foot, a peroneal artery, an anterior tibial artery, and a posterior tibial artery and at least one other blood vessel or other two blood vessels.

Further, the limb to which the hemostasis assisting tool is applicable may be a leg instead of an arm, for example. For example, the hemostasis assisting tool may be used to promote hemostasis on a puncture site formed in either one of an anterior tibial artery, a posterior tibial artery, and a peroneal artery that are branched from a popliteal artery and that extend parallel to each other. Further, the hemostasis assisting tool may be used to promote hemostasis on a puncture site formed in either one of a lateral plantar artery and a medial plantar artery that are branched from a posterior tibial artery in a leg and that extend parallel to each other, or a dorsal artery of foot branched from an anterior tibial artery.

Further, according to the first embodiment, the temperature adjusting portion 30 of the hemostasis assisting tool 10 has both the heating portion 31 and the cooling portion 32. However, the temperature adjusting portion 30 may have only one of the heating portion 31 and the cooling portion 32. In such a case, the volume of a blood flow (the amount of a blood coagulation factor) in the radial artery V1 is increased by expanding the radial artery V1 and/or the peripheral blood vessel V3 of the radial artery V1 with the heating portion 31 or contracting the ulnar artery V2 and/or the peripheral blood vessel V4 of the ulnar artery V2 with the cooling portion 32. Therefore, the hemostasis on the puncture site P formed in the radial artery V1 can be promoted.

Further, the hemostasis assisting tool may be free of a mount portion and may have a heating portion and a cooling portion directly mounted on the forearm, for example. Further, the hemostasis assisting tool is not limited to one to be mounted on an arm, and may be one to be mounted on a leg or a foot, for example.

The detailed description above describes embodiments of a hemostasis promoting method and a hemostasis assisting tool representing examples of the inventive hemostasis promoting method and hemostasis assisting tool disclosed here. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A hemostasis promoting method for promoting hemostasis at a puncture site formed in a first blood vessel, the first blood vessel and a second vessel branching from a blood vessel in a limb and extending parallel to each other, the hemostasis promoting method comprising:
making a temperature of a body surface around the first blood vessel and/or a peripheral blood vessel of the first blood vessel higher than a temperature of a body surface around the second blood vessel and/or a peripheral blood vessel of the second blood vessel such that the temperature of the first blood vessel and/or the peripheral blood vessel of the first blood vessel is higher than the temperature of the second blood vessel and/or the peripheral blood vessel of the second blood vessel.

2. The hemostasis promoting method according to claim 1, wherein the making of the temperature of the body surface around the first blood vessel and/or the peripheral blood vessel of the first blood vessel higher than the temperature of the body surface around the second blood vessel and/or the peripheral blood vessel of the second blood vessel comprises: i) heating the body surface around the first blood vessel and/or the peripheral blood vessel of the first blood vessel; or ii) cooling the body surface around the second blood vessel and/or the peripheral blood vessel of the second blood vessel.

3. The hemostasis promoting method according to claim 1, wherein the making of the temperature of the body surface around the first blood vessel and/or the peripheral blood vessel of the first blood vessel higher than the temperature of the body surface around the second blood vessel and/or the peripheral blood vessel of the second blood vessel comprises: i) heating the body surface around the first blood vessel and/or the peripheral blood vessel of the first blood vessel: and ii) cooling the body surface around the second blood vessel and/or the peripheral blood vessel of the second blood vessel.

4. The hemostasis promoting method according to claim 3, wherein the making of the temperature of the body surface around the first blood vessel and/or the peripheral blood vessel of the first blood vessel higher than the temperature of the body surface around the second blood vessel and/or the peripheral blood vessel of the second blood vessel comprises heating the body surface around a portion of the first blood vessel that is positioned more to an upstream side of the first blood vessel than the puncture site.

5. The hemostasis promoting method according to claim 1, wherein the making of the temperature of the body surface around the first blood vessel and/or the peripheral blood vessel of the first blood vessel higher than the temperature of the body surface around the second blood vessel and/or the peripheral blood vessel of the second blood vessel comprises heating the body surface around the first blood vessel and/or the peripheral blood vessel of the first blood vessel to increase the temperature of the body surface around the first blood vessel and/or the peripheral blood vessel of the first blood vessel to a range of 33° C. to 37° C.

6. The hemostasis promoting method according to claim 1, wherein the making of the temperature of the body surface around the first blood vessel and/or the peripheral blood vessel of the first blood vessel higher than the temperature of the body surface around the second blood vessel and/or the peripheral blood vessel of the second blood vessel comprises cooling the body surface around the second blood vessel and/or the peripheral blood vessel of the second blood vessel to reduce the temperature of the body surface around the second blood vessel and/or the peripheral blood vessel of the second blood vessel to a range of 10° C. to 27° C.

7. A hemostasis assisting tool mountable on a limb of a living body for assisting hemostasis at a puncture site in a first blood vessel that extends parallel to a second blood vessel, with both the first and second blood vessels branching from a blood vessel in the limb, the hemostasis assisting tool comprising:
a temperature adjusting portion for making a temperature of a body surface around the first blood vessel and/or a peripheral blood vessel of the first blood vessel higher than a temperature of a body surface around the second blood vessel and/or a peripheral blood vessel of the second blood vessel such that the temperature of the first blood vessel and/or the peripheral blood vessel of the first blood vessel is higher than the temperature of the second blood vessel and/or the peripheral blood vessel of the second blood vessel.

8. The hemostasis assisting tool according to claim 7, wherein the temperature adjusting portion includes: i) a heating portion for heating the body surface around the first blood vessel and/or the peripheral blood vessel of the first blood vessel: or ii) a cooling portion for cooling the body surface around the second blood vessel and/or the peripheral blood vessel of the second blood vessel.

9. The hemostasis assisting tool according to claim 7, wherein the temperature adjusting portion includes: i) a heating portion for heating the body surface around the first blood vessel and/or the peripheral blood vessel of the first blood vessel; and ii) a cooling portion for cooling the body surface around the second blood vessel and/or the peripheral blood vessel of the second blood vessel.

10. The hemostasis assisting tool according to claim 9, wherein
the heating portion includes a first housing portion housing a heat generating substance that generates heat by reacting with a first fluid and a first injection starting mechanism for starting injection of the first fluid into the first housing portion, and
the cooling portion has a second housing portion housing a heat absorbing substance that absorbs heat by reacting with a second fluid and a second injection starting mechanism for starting injection of the second fluid into the second housing portion.

11. The hemostasis assisting tool according to claim 9, wherein
the heating portion includes a heating sheet configured to be affixed to the body surface around the first blood vessel and/or the peripheral blood vessel of the first blood vessel, for heating the body surface, and
the cooling portion has a cooling sheet configured to be affixed to the body surface around the second blood vessel and/or the peripheral blood vessel of the second blood vessel, for cooling the body surface.

12. The hemostasis assisting tool according to claim 11, further comprising:
a heat transfer restraining portion, disposed between the heating portion and the cooling portion, that restrains heat transfer between the heating portion and the cooling portion.

13. The hemostasis assisting tool according to claim 11, further comprising:
a covering that covers surfaces of the heating portion and the cooling portion that face the limb when the hemostasis assisting tool is mounted on the limb of the living body.

14. The hemostasis assisting tool according to claim 13, wherein the covering includes a pouch having an interior configured to be filled with a gas and an injection port communicating with the interior of the pouch to permit injection of the gas into the interior of the pouch using an injection instrument and prevent injected gas from leaking out of the interior of the pouch.

15. The hemostasis assisting tool according to claim 9, further comprising a mount portion to which the temperature adjusting portion is fixed, the mount portion including a through opening that passes through the mount portion, the mount portion being mountable on the limb of the living body so that the puncture site is exposed through the through opening.

16. The hemostasis assisting tool according to claim 7, wherein the temperature adjusting portion includes a heating portion for heating the body surface around the first blood vessel and/or the peripheral blood vessel of the first blood vessel, the heating portion being configured to be disposed on the body surface around a portion of the first blood vessel that is positioned more to an upstream side of the first blood vessel than the puncture site.

17. A hemostasis promoting method for promoting hemostasis at a puncture site formed in a first blood vessel of a limb of a living body, the first blood vessel and a second vessel branching from a blood vessel in the limb and extending parallel to one another, the hemostasis promoting method comprising:
positioning a limb mount on the limb of the patient adjacent the puncture site so that the limb mount encircles the limb of the living body;
after the positioning of the limb mount on the limb of the living body adjacent the puncture site: i) applying heat to the body surface of the limb around the first blood vessel and/or a peripheral blood vessel of the first blood vessel to increase a temperature of the body surface around the first blood vessel and/or a peripheral blood vessel of the first blood vessel; and/or ii) applying cooling to the body surface of the limb around the second blood vessel and/or a peripheral blood vessel of the second blood vessel to decrease the temperature of the body surface around the second blood vessel and/or a peripheral blood vessel of the second blood vessel so that the temperature of the first blood vessel and/or the peripheral blood vessel of the first blood vessel is greater than the temperature of the second blood vessel and/or the peripheral blood vessel of the second blood vessel to thus cause a volume of blood flow in the first blood vessel to be increased and an amount of a blood coagulation factor in the first blood vessel to be increased so that hemostasis on the puncture site is promoted; and
the applying of the heat and/or the applying of the cooling being accomplished by a part on the limb mount.

18. The hemostasis promoting method according to claim 17, wherein the positioning of the limb mount on the limb of the living body comprises: i) positioning a tubular member on the limb of the living body; ii) positioning a glove on a hand of the living limb so that the glove encloses at least a part of fingers of the hand; or iii) wrapping a band around the limb.

19. The hemostasis promoting method according to claim 17, wherein the part on the limb mount is a heater fixed to the limb mount to apply the heat to the body surface of the limb around the first blood vessel and/or the peripheral blood vessel of the first blood vessel, and a cooler fixed to the limb mount to apply the cooling to the body surface of the limb around the second blood vessel and/or the peripheral blood vessel of the second blood vessel.

20. The hemostasis promoting method according to claim 17, wherein the applying of the heat to the body surface of the limb around the first blood vessel and/or a peripheral blood vessel of the first blood vessel and/or the applying of the cooling to the body surface of the limb around the second blood vessel and/or a peripheral blood vessel of the second blood vessel includes applying both the heat to the body surface of the limb around the first blood vessel and/or a peripheral blood vessel of the first blood vessel and the cooling to the body surface of the limb around the second blood vessel and/or a peripheral blood vessel of the second blood vessel.
